(19) United States
(12) Patent Application Publication
(10) Pub. No.: US 2026/0079120 A1
(43) Pub. Date: Mar. 19, 2026

(54) APPARATUS AND METHOD FOR INSPECTING X-RAY

(71) Applicant: ELECTRONICS AND TELECOMMUNICATIONS RESEARCH INSTITUTE, Daejeon (KR)

(72) Inventor: Jin Woo JEONG, Daejeon (KR)

(21) Appl. No.: 19/004,677

(22) Filed: Dec. 30, 2024

(30) Foreign Application Priority Data
Sep. 19, 2024 (KR) 10-2024-0126959
Dec. 23, 2024 (KR) 10-2024-0194685

(51) Int. Cl. G01N 23/044; G01N 23/083

(52) U.S. Cl. CPC G01N 23/044; G01N 23/083; G01N 2223/20; G01N 2223/3303; G01N 2223/401

(57) ABSTRACT

The present invention relates to an X-ray inspection apparatus and method. The X-ray inspection apparatus according to one embodiment includes a transfer unit configured to transfer a subject in a first direction, a plurality of X-ray sources arranged to be spaced apart from each other in a second direction intersecting the first direction and configured to radiate X-rays toward the subject, an X-ray control unit configured to control an arrangement of the plurality of X-ray sources, a detection unit positioned below the transfer unit and configured to detect an X-ray transmission image passing of the X-rays through the subject, and an image processing unit configured to generate an X-ray image based on the X-ray transmission image detected by the detection unit.

### CROSS-REFERENCE TO RELATED APPLICATIONS

This application claims priority to and the benefit of Korean Patent Application No. 10-2024-0126959, filed Sep. 19, 2024 and Korean Patent Application No. 10-2024-0194685, filed Dec. 23, 2024, the disclosures of which are incorporated herein by reference in their entireties.

### BACKGROUND

### 1. Field of the Invention

The present invention relates to an X-ray inspection apparatus and method allowing a subject to be inspected using a plurality of X-ray sources.

### 2. Description of Related Art

An X-ray nondestructive inspection is being performed to determine whether there are any internal defects in products produced in factories. Food is inspected using relatively simple two-dimensional X-ray images to detect foreign materials in packaged products, and in the case of products with fine and complex structures, such as information and communications technology (ICT) and secondary battery components, a three-dimensional reconstruction X-ray image inspection is required. A nondestructive inspection using X-rays may be typically performed using three-dimensional computerized tomography (CT) equipment, but since general equipment takes a very long time to perform an inspection, it is impossible to perform an in-line inspection in real time.

The background of the present invention is disclosed in Korean Patent Publication No. 10-1685005 (published on Dec. 13, 2016).

### SUMMARY OF THE INVENTION

The present invention is directed to providing an X-ray inspection apparatus and method allowing a two-dimensional or three-dimensional image of a subject, which is moved by a transfer unit, to be acquired in real time using a plurality of X-ray sources.

According to an aspect of the present invention, there is provided an X-ray inspection apparatus including a transfer unit configured to transfer a subject in a first direction, a plurality of X-ray sources arranged to be spaced apart from each other in a second direction intersecting the first direction and configured to radiate X-rays toward the subject, an X-ray control unit configured to control an arrangement of the plurality of X-ray sources, a detection unit positioned below the transfer unit and configured to detect an X-ray transmission image of the X-rays passing through the subject, and an image processing unit configured to generate an X-ray image based on the X-ray transmission image detected by the detection unit.

The plurality of X-ray sources may be sequentially driven one by one under a control of the X-ray control unit to radiate the X-rays, and the detection unit may acquire the X-ray transmission image in synchronization with X-ray radiation of each X-ray source.

The X-ray control unit may control an X-ray radiation time and an X-ray amount of each X-ray source.

The X-ray control unit may control the plurality of X-ray sources to be disposed in at least one of a parallel arrangement, a plane rotation arrangement, a spatial rotation arrangement, and an enlarged spatial rotation arrangement.

In the parallel arrangement, the plurality of X-ray sources may be disposed perpendicular to an X-ray detection surface of the detection unit and disposed parallel to each other at a set interval.

In the plane rotation arrangement, the plurality of X-ray sources may be disposed perpendicular to an X-ray detection surface of the detection unit, and each X-ray source may be rotated to face a center of the X-ray detection surface.

In the spatial rotation arrangement, the plurality of X-ray sources may be rotated by a first rotation angle in a moving direction of the subject and rotated by a second rotation angle in a direction perpendicular to the moving direction of the subject, focal points of the plurality of X-ray sources may be disposed at equal intervals with respect to the moving direction of the subject, and a center of the X-rays radiated by each X-ray source may be disposed to face an X-ray detection surface of the detection unit.

The second rotation angle may be determined by at least one of an X-ray emission angle, a distance between the X-ray source and the subject, a distance between the subject and the detection unit, and a field of view (FOV) of the subject, and an area of the detection unit.

In the enlarged space rotation arrangement, the plurality of X-ray sources may be disposed to be rotated by the first rotation angle in the moving direction of the subject and rotated by a third rotation angle in the direction perpendicular to the moving direction of the subject, and a plurality of detection units may be disposed in the direction perpendicular to the moving direction of the subject, wherein the third rotation angle has a larger value than the second rotation angle.

The plurality of X-ray sources may operate in at least one of a stitching mode, a region of interest (ROI) mode, and a tomography mode.

When the plurality of X-ray sources operate in the stitching mode, the plurality of X-ray sources may be sequentially driven in the first direction as the subject moves in the first direction, the detection unit may detect a plurality of partial X-ray transmission images as the X-ray sources are sequentially driven, and the image processing unit may image-process the plurality of partial X-ray transmission images detected by the detection unit to generate one or more high-resolution X-ray images.

When the plurality of X-ray sources operate in the ROI mode, as the subject moves in the first direction, an X-ray source, to which the FOV of the subject reaches, among the plurality of X-ray sources, may radiate the X-rays,
the detection unit may detect an X-ray transmission image of the FOV, and the image processing unit may image-process the X-ray transmission image of the FOV detected by the detection unit to generate one or more X-ray images of the FOV.

When the plurality of X-ray sources operate in the tomography mode, the plurality of X-ray sources may be sequentially driven in the first direction to radiate the X-rays at different angles a plurality of times as the subject moves in the first direction, the detection unit may detect X-ray transmission images at different angles, and the image processing unit may image-process the X-ray transmission image detected through the detection unit to generate a tomogram.

According to an aspect of the present invention, there is provided an X-ray inspection method including arranging, by an X-ray control unit, a plurality of plurality of X-ray sources in an arrangement structure selected by a user, sequentially driving the plurality of X-ray sources under a control of the X-ray control unit to radiate X-rays onto a subject moved by a transfer unit, detecting, by a detection unit, an X-ray transmission image of the X-rays passing through the subject, and generating, by an image processing unit, an X-ray image based on the X-ray transmission image detected by the detection unit.

In the arranging of the plurality of X-ray sources, the X-ray control unit may control the plurality of X-ray sources to be disposed in at least one of a parallel arrangement, a plane rotation arrangement, a spatial rotation arrangement, and an enlarged spatial rotation arrangement.

In the parallel arrangement, the plurality of X-ray sources may be disposed perpendicular to an X-ray detection surface of the detection unit and disposed parallel to each other at a set interval.

In the plane rotation arrangement, the plurality of X-ray sources may be disposed perpendicular to an X-ray detection surface of the detection unit, and each X-ray source may be rotated to face a center of the X-ray detection surface.

In the spatial rotation arrangement, the plurality of X-ray sources may be rotated by a first rotation angle in a moving direction of the subject and rotated by a second rotation angle in a direction perpendicular to the moving direction of the subject, focal points of the plurality of X-ray sources may be disposed at equal intervals with respect to the moving direction of the subject, and a center of the X-rays radiated by each X-ray source may be disposed to face an X-ray detection surface of the detection unit.

In the enlarged space rotation arrangement, the plurality of X-ray sources may be disposed to be rotated by the first rotation angle in the moving direction of the subject and rotated by a third rotation angle in the direction perpendicular to the moving direction of the subject, and a plurality of detection units may be disposed in the direction perpendicular to the moving direction of the subject, wherein the third rotation angle has a larger value than the second rotation angle.

In the radiating of the X-rays, the plurality of X-ray sources may operate in at least one of a stitching mode, a region of interest (ROI) mode, and a tomography mode.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a schematic block diagram of a configuration of an X-ray inspection apparatus according to one embodiment of the present invention;

FIG. 2 is a diagram for describing an X-ray source unit according to one embodiment of the present invention;

FIG. 3A is a side view for describing a parallel arrangement of X-ray sources according to one embodiment of the present invention;

FIG. 3B is a plan view for describing the parallel arrangement of the X-ray sources according to one embodiment of the present invention.

FIGS. 4A-4C show exemplary diagrams for describing a stitching mode according to one embodiment of the present invention;

FIGS. 5A-5C show exemplary diagrams for describing a region of interest mode according to one embodiment of the present invention;

FIGS. 6A-6B show exemplary diagrams for describing a tomography mode according to one embodiment of the present invention;

FIG. 7A is a side view for describing a planar rotation arrangement of the X-ray sources according to one embodiment of the present invention;

FIG. 7B is a plan view for describing the planar rotation arrangement of the X-ray sources according to one embodiment of the present invention;

FIG. 8 is an exemplary diagram for describing the operation of the X-ray sources in the planar rotation arrangement according to one embodiment of the present invention;

FIG. 9A is a side view for describing a spatial rotation arrangement of the X-ray sources according to one embodiment of the present invention;

FIG. 9B is a plan view for describing the spatial rotation arrangement of the X-ray sources according to one embodiment of the present invention;

FIG. 10 is an exemplary diagram for describing a second rotation angle according to one embodiment of the present invention;

FIG. 11A is a side view for describing an enlarged spatial rotation arrangement of the X-ray sources according to one embodiment of the present invention; and

FIG. 11B is a plan view for describing the enlarged spatial rotation arrangement of the X-ray sources in one embodiment of the present invention.

### DETAILED DESCRIPTION OF EXEMPLARY EMBODIMENTS

Hereinafter, embodiments of an X-ray inspection apparatus and method according to the present invention will be described with reference to the accompanying drawings. The drawings are not necessarily to scale and in some instances, proportions may have been exaggerated in order to clearly illustrate features of the embodiments. Further, the terms to be described below are terms defined in consideration of functions in the present invention and thus may vary according to intentions of users or operators or customs. Accordingly, the definitions of such terms should be made based on the content throughout the specification.

To obtain a three-dimensional (3D) X-ray image, an X-ray source and a sensor are rotated around a subject to obtain a two-dimensional (2D) projection image at various angles, and then the 2D projection image is mathematically reconstructed to obtain a 3D image. A method of acquiring the 3D image necessarily requires mechanical rotational photographing, which makes rapid inspection impossible and decreases a production speed. Typically, products manufactured in factories often linearly move on a conveyor belt in each process operation. Unlike a method of reconstructing a 3D X-ray image by rotating an X-ray source and a detection unit (sensor) around a subject, when a product moving on a conveyor belt during a production process can be inspected without interference, a fast production speed can be ensured.

Accordingly, the present invention proposes a technology capable of acquiring a 2D or 3D image of a subject, which linearly moves on a conveyor belt, in real time using a plurality of X-ray sources.

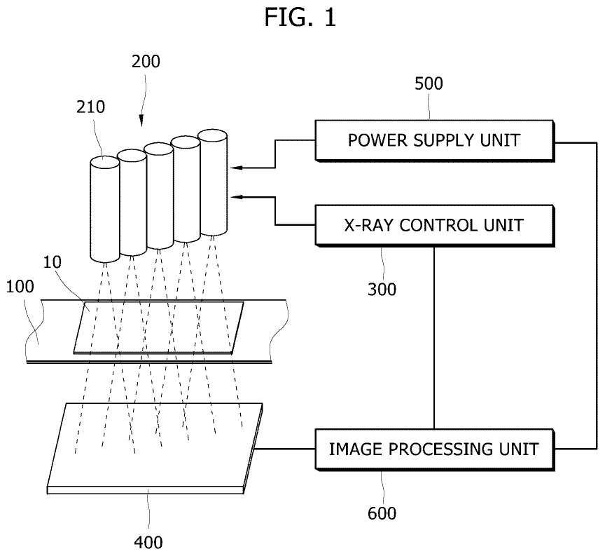

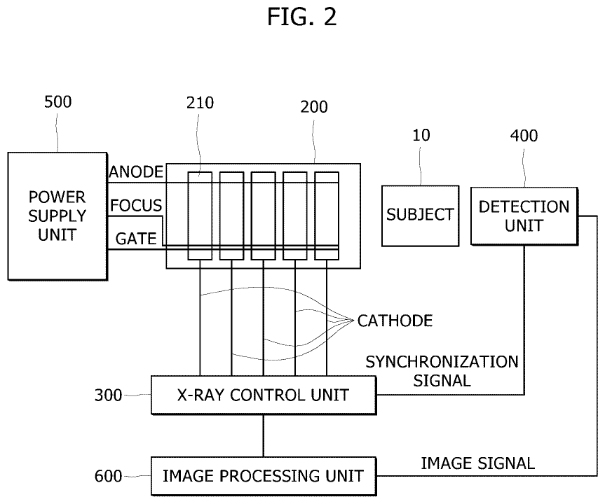

FIG. 1 is a schematic block diagram illustrating a configuration of an X-ray inspection apparatus according to one embodiment of the present invention. FIG. 2 is a diagram for describing an X-ray source unit according to one embodiment of the present invention.

Referring to FIG. 1, the X-ray inspection apparatus according to one embodiment of the present invention may include a transfer unit 100, an X-ray source unit 200, an X-ray control unit 300, a detection unit 400, a power supply unit 500, and an image processing unit 600.

The transfer unit 100 may be configured to transfer a subject 10 in a first direction. According to an example, the transfer unit 100 may include a support member (not shown), rollers (not shown), and a conveyor belt (not shown). The rollers may be rotatably connected to the support and may be disposed to be spaced apart from each other in the first direction. The conveyor belt may be provided to be wrapped around the rollers. The conveyor belt may support the subject 10. The conveyor belt may rotate according to the rotation of the rollers. The subject 10 may be transferred in the first direction according to the rotation of the conveyor belt.

The X-ray source unit 200 may be disposed above the transfer unit 100. The X-ray source unit 200 may generate X-rays to radiate the X-rays onto the subject 10.

The X-ray source unit 200 may generate X-rays by applying a high voltage between a negative electrode and a positive electrode of a vacuum tube included therein. The intensity of X-rays output by an X-ray source 210 may vary according to a tube voltage, a tube current, and a pulse shape of an X-ray tube applied to the vacuum tube.

The X-ray source unit 200 may include a plurality of X-ray sources 210.

The plurality of X-ray sources 210 may be field emission type X-ray sources.

As shown in FIG. 2, the plurality of X-ray sources 210 have cathodes at which electric field-emitting sources are disposed, and electrodes (anodes, focus electrodes, gate electrodes, and the like) of the X-ray sources 210 may be commonly connected to the same power supply unit 500 to receive power.

The plurality of X-ray sources 210 may be arranged to be spaced apart from each other in a second direction intersecting with the first direction, which is a moving direction of the subject 10, and may radiate X-rays toward the subject 10.

The radiation (emission) of X-rays from the plurality of X-ray sources 210 may be controlled by the X-ray control unit 300 connected to each cathode. In this case, the X-ray control unit 300 may control a radiation (emission) time and a radiation amount of X-rays by controlling a current using a high-voltage metal oxide silicon field effect transistor (MOSFET) or the like.

The X-ray control unit 300 may be connected to the image processing unit 600 together with the detection unit 400 to control X-ray radiation (emission) of the X-ray source 210 in a synchronization manner. In this case, the X-ray control unit 300 may control the plurality of X-ray sources 210 to simultaneously emit X-rays, but generally, only one X-ray source 210 may be controlled to be driven at a time such that an X-ray image emitted by one X-ray source 210 is obtained.

The X-ray control unit 300 may control the arrangement of the plurality of X-ray sources 210. The X-ray control unit 300 may change positions of the X-ray source 210 and the detection unit 400 with respect to the subject 10 to adjust an image magnification or the like according to the purpose of an inspection. In this case, the X-ray control unit 300 may control the plurality of X-ray sources 210 to be disposed in at least one of a parallel arrangement, a plane rotation arrangement, a spatial rotation arrangement, and an enlarged spatial rotation arrangement. The arrangement of the X-ray sources will be described in detail below.

The X-ray control unit 300 may actively control an X-ray dose output from the X-ray source unit 200 based on the movement of the subject 10 to radiate X-rays onto the subject 10.

The X-ray control unit 300 may be connected to the X-ray source unit 200 and may control the X-ray source unit 200. For example, the X-ray control unit 300 may synchronize and control the X-ray source unit 200 such that X-rays may be radiated onto the subject 10 in a preset direction. The X-ray control unit 300 may be a concept encompassing a computer system on which an X-ray source unit driving algorithm is installed.

Although not shown in the drawing, the X-ray control unit 300 may include a processor for executing instructions and an internal memory. The processor included in the X-ray control unit 300 may include a graphics processing unit (GPU) for processing graphics. The processor may be implemented as a system-on-chip (SoC) into which a core and a GPU are integrated. The processor may include a single core, a dual core, a triple core, a quad core, and a multiple core thereof. The processor may execute instructions to perform control operations on the X-ray source unit 200 and the detection unit 400.

The internal memory included in the X-ray control unit 300 may include a random access memory (RAM) that stores signals or data input from the outside of the X-ray inspection apparatus or is used as a storage area for various operations performed in the X-ray inspection apparatus. In addition, the internal memory included in the X-ray control unit 300 may include a read-only memory (ROM) that stores a control program for controlling the X-ray source unit 200 and the detection unit 400 and instructions executed by the processor.

Meanwhile, although not shown in FIG. 1, the X-ray inspection apparatus may further include an optical position sensor (not shown), such as a laser, to detect a position of the subject 10 suitable for acquiring an X-ray image. In addition, the X-ray inspection apparatus may acquire an X-ray image in accordance with an expected entry time of the subject 10 to detect a position of the subject 10 and a timing based on acquired image information.

The detection unit 400 may be disposed below the transfer unit 100. The detection unit 400 may detect X-rays that have passed through the subject 10 and may acquire an X-ray transmission image of the subject 10. Specifically, the detection unit 400 may convert X-rays that have passed through the subject 10 into an electrical signal and may amplify and convert the converted electrical signal into an X-ray transmission image. The detection unit 400 may be provided in a flat plate shape, but the present invention is not limited thereto. One or more detection units 400 may be provided in a linear form or the like to prevent distortion of X-rays input to the detection unit 400.

The image processing unit 600 may be configured to receive X-ray transmission images from the detection unit 400. The image processing unit 600 receives X-ray transmission images from the detection unit 400 and may reconstruct the received X-ray transmission images into a 3D image. In this case, the image processing unit 600 may be a concept encompassing a computer system on which an image processing algorithm is installed.

The image processing unit 600 may include a processor configured to execute instructions and an internal memory. The processor included in the image processing unit 600 may include a GPU for processing graphics. The processor may be implemented as an SoC into which a core and a GPU are integrated. The processor may include a single core, a dual core, a triple core, a quad core, and a multiple core thereof. The processor may execute instructions to perform control operations on the X-ray source unit 200 and the detection unit 400.

The internal memory included in the image processing unit 600 may include a RAM that stores signals or data input from the outside of the X-ray inspection apparatus or is used as a storage area for various operations performed in the X-ray inspection apparatus. In addition, the internal memory included in the image processing unit 600 may include a ROM that stores a program for reconstructing an X-ray transmission image into a 3D mage and instructions executed by the processor.

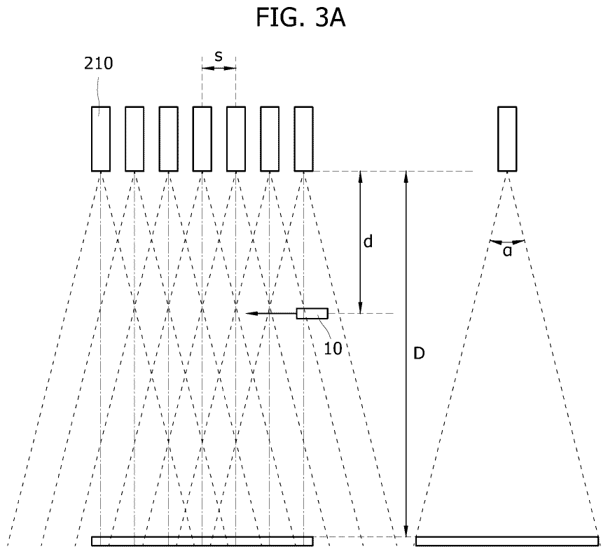

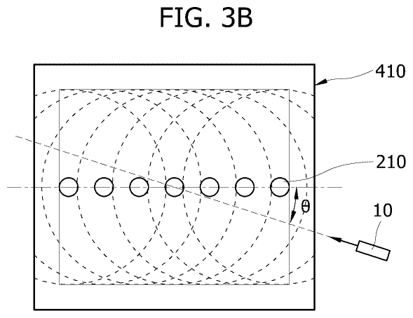

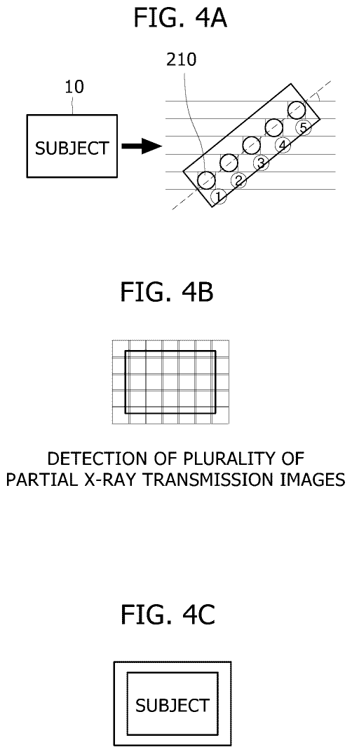

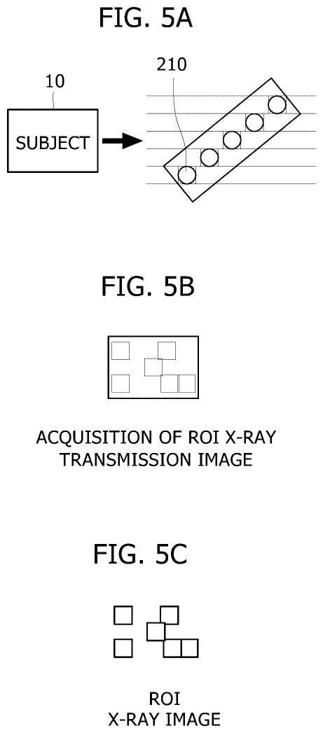

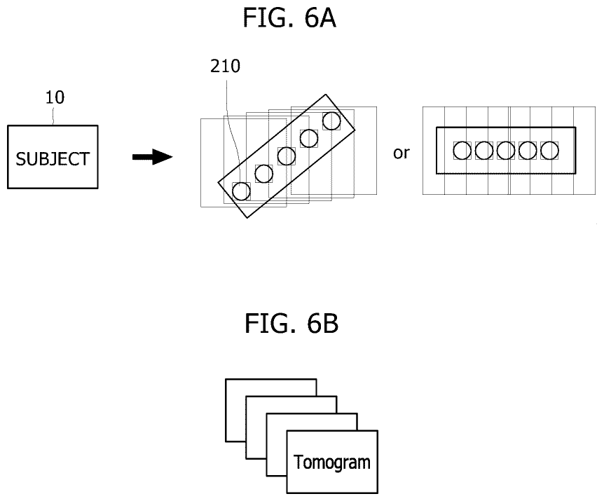

FIG. 3A is a side view for describing a parallel arrangement of the X-ray sources according to one embodiment of the present invention. FIG. 3B is a plan view for describing the parallel arrangement of the X-ray sources according to one embodiment of the present invention. FIG. 4 shows exemplary diagrams for describing a stitching mode according to one embodiment of the present invention. FIG. 5 shows exemplary diagrams for describing a region of interest (ROI) mode according to one embodiment of the present invention. FIG. 6 shows exemplary diagrams for describing a tomography mode according to one embodiment of the present invention.

Referring to FIGS. 3A and 3B, the parallel arrangement may be applied to a structure in which the plurality of X-ray sources 210 are disposed perpendicular to an X-ray detection surface 410 of the detection unit 400 and parallel to each other at constant intervals. In this case, X-ray radiation (emission) center lines may be disposed perpendicular to the X-ray detection surface 410 and parallel to each other. In this case, the plurality of X-ray sources 210 are sequentially driven one by one to generate X-rays, and the detection unit 400 may acquire an X-ray transmission image in synchronization with X-ray emission. In the parallel arrangement, the plurality of X-ray sources 210 may be disposed at a certain angle θ (arrangement angle) with respect to a moving direction of the subject 10, thereby adjusting a vertical distance by which the subject moves.

The plurality of X-ray sources 210 may operate in three image acquisition modes shown in FIGS. 4A to 6B. Here, the image acquisition modes may include a stitching mode, an ROI mode, and a tomography mode.

The stitching mode may correspond to a method of acquiring a high-resolution X-ray image in a wide area by combining X-ray transmission images acquired at different positioned using the plurality of X-ray sources 210 under conditions in which, due to a high magnification of the subject 10, an area to be photographed is narrow. In the stitching mode, the X-ray sources 210 may be sequentially driven as the subject 10 moves in a moving direction as shown in FIGS. 4A-4C. In this case, the X-ray sources 210 may repeatedly perform sequential driving until the subject 10 exits an inspection area. For example, as shown in FIG. 4A, X-ray source \textcircled{1}, X-ray source \textcircled{2}, X-ray source \textcircled{3}, X-ray source \textcircled{4}, and X-ray source \textcircled{5} may be sequentially driven. As shown in FIG. 4B, the detection unit 400 may detect a plurality of partial X-ray transmission images as the X-ray sources 210 are sequentially driven, and as shown in FIG. 4C, the image processing unit 600 may image-process the plurality of partial X-ray transmission images detected through the detection unit 400 to generate one or more high-resolution X-ray images.

The X-ray source 210 may be disposed at a certain angle with respect to a moving direction of the subject 10 to adjust a vertical distance by which the subject moves. For example, when the angle θ (arrangement angle) between an arrangement direction of the X-ray sources 210 and the moving direction of the subject is increased, a distance between the X-ray sources in a vertical direction in which the subject moves may be increased. In this case, the maximum distance between the X-ray sources 210 may be a distance between the physically fixed X-ray sources 210. When the angle θ (arrangement angle) between the arrangement direction of the X-ray sources 210 and the moving direction of the subject is decreased, the distance between the X-ray sources 210 may be decreased, and the minimum distance may be “0.” In the case of the wide subject 10, the angle θ (arrangement angle) between the arrangement direction of the X-ray sources 210 and the moving direction of the subject may be increased, and in the case of the narrow subject 10, the angle θ (arrangement angle) between the arrangement direction of the X-ray sources 210 and the moving direction of the subject may be decreased or the number of X-ray sources 210 may be increased to compensate for an uncovered area.

The ROI mode may be applied when a full image of the subject is not required, or an inspection speed is to be increased. In the ROI mode, as shown in FIG. 5A, the X-ray source 210 may be disposed similarly to the stitching mode so that, when a field of view (FOV) of the subject 10 reaches a radiation area of the X-ray source 210, the X-ray source 210 may radiate X-rays. As shown in FIG. 5B, the detection unit 400 may detect an X-ray transmission image of the FOV, and as shown in FIG. 5C, the image processing unit 600 may image-process the X-ray transmission image of the FOV detected through the detection unit 400 to obtain one or more X-ray images of the FOV.

In the case of a conventional inspection apparatus in which one X-ray source is disposed, in order to inspect an FOV, the subject 10 or the X-ray source 210 and detection unit 400 were moved a plurality of times in a 3D space to capture an image. However, when the present invention is applied, there is an effect in which an in-line inspection may be performed in real time without interference of the subject 10 moving on the transfer unit 100. In the ROI mode, the arrangement angle of the X-ray sources 210 and the number of the X-ray sources may be adjusted according to the size of the subject and the position of the FOV as in the stitching mode.

The tomography mode may be applied to sequentially drive the plurality of X-ray sources 210 with respect to the moving subject 10 and obtain a tomogram using an X-ray transmission image acquired through the detection unit 400. In tomography mode, when the subject 10 moves in a state in which the plurality of X-ray sources 210 and the detection unit 400 are fixed, X-rays may be radiated onto the subject 10 at different angles as the X-ray sources 210 are sequentially driven when the subject 10 moves, and the detection unit 400 may detect an X-ray transmission image at various angles. In the tomography mode, as shown in FIG. 6A, X-rays may be radiated onto the moving subject 10 at different angles a plurality of times as the X-ray sources 210 are sequentially driven, and the detection unit 400 may detect a wide-angle X-ray transmission image. The image processing unit 600 may process the X-ray transmission image detected through the detection unit 400 to acquire a tomogram as shown in FIG. 6B.

Meanwhile, when the plurality of X-ray sources 210 are disposed in parallel, since an X-ray emission angle α is finite, when a distance between the X-ray sources 210 is increased in a parallel arrangement configuration of the X-ray sources increases, the entire X-ray detection surface 410 of the detection unit 400 may not be covered. Such a problem may be more highlighted, in particular, at a high magnification at which a distance between the X-ray source 210 and the subject 10 is smaller than a distance between the subject 10 and the detection unit 400.

Accordingly, in the present invention, each X-ray source 210 is disposed to be rotated to face a center of the X-ray detection surface 410, thereby enabling more X-ray transmission images of the moving subject 10 to be acquired even under high magnification conditions.

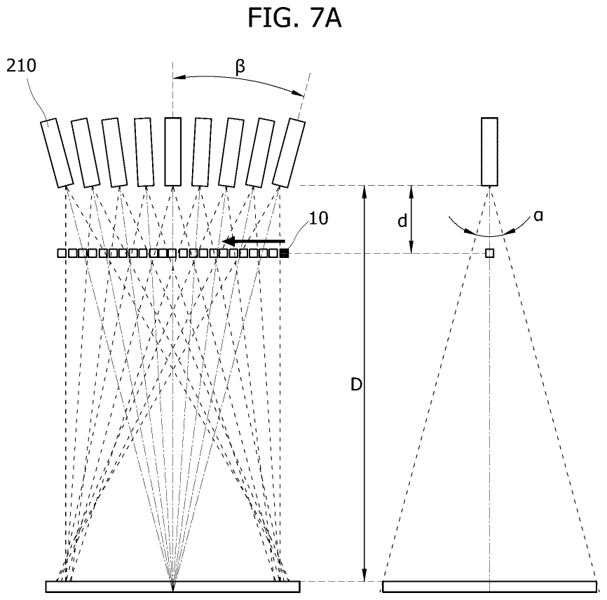

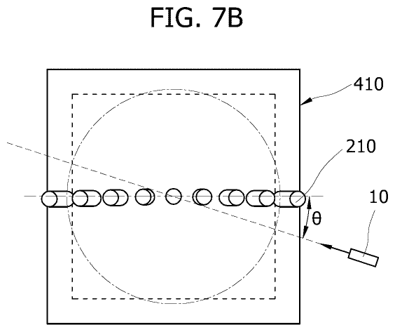

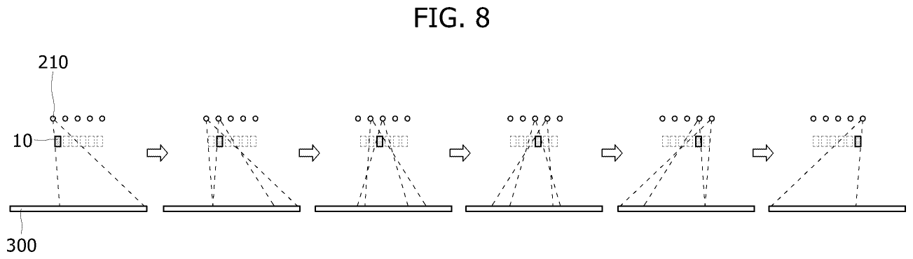

FIG. 7A is a side view for describing a planar rotation arrangement of the X-ray sources according to one embodiment of the present invention. FIG. 7B is a plan view for describing the planar rotation arrangement of the X-ray sources according to one embodiment of the present invention. FIG. 8 is an exemplary diagram for describing the operation of the X-ray sources in the planar rotation arrangement according to one embodiment of the present invention.

Referring to FIGS. 7A and 7B, the planar rotation arrangement of the X-ray sources 210 may be applied to a structure in which the plurality of X-ray sources 210 are disposed perpendicular to the X-ray detection surface 410 of the detection unit 400, and each X-ray source 210 is disposed to be rotated to face the center of the X-ray detection surface 410. In this case, the X-ray source 210 may be rotated by the maximum rotation angle β. In the planar rotation arrangement, the plurality of X-ray sources 210 may be disposed at a certain angle θ (arrangement angle) with respect to a moving direction of the subject 10.

When the X-ray sources 210 are disposed in the planar rotation arrangement, as shown in FIG. 8, for the plurality of X-ray sources 210 that emit X-rays toward the detection unit 400 and the subject 10 passing between the X-ray sources 210, as a position of the subject 10 changes over time, only some of the X-ray sources 210 adjacent to the subject 10 among the plurality of X-ray sources 210 may be driven so that an X-ray transmission image may be acquired. In this case, the X-ray control unit 300 may select and drive the X-ray source 210 necessary for image acquisition in consideration of at least one of a speed of the moving subject 10, an X-ray driving cycle, an image acquisition cycle of the detection unit, and a degree by which a transmission image is covered. In FIG. 8, a micro-displacement of the subject 10 is ignored, but actually, while the subject 10 moves, many X-ray transmission images at various angles may be acquired as adjacent X-ray sources 210 are repeatedly, rapidly, sequentially driven, thereby enabling tomogram synthesis.

In the case of the planar rotation arrangement, the arrangement angle θ between the arrangement of the X-ray sources and the moving direction of the subject 10 may be changed and adjusted between 0 degrees and 90 degrees to effectively acquire an image, and the arrangement of each X-ray source 210 may be adjusted to effectively acquire an X-ray transmission image.

Meanwhile, both the parallel arrangement and the planar rotation arrangement of the X-ray sources 210 have a form in which the plurality of X-ray sources 210 are fixed onto one plane. In this case, an X-ray transmission image perpendicular to a plane cannot be obtained, resulting in insufficient information when 3D images are synthesized. To overcome such shortcomings, the X-ray source 210 may be rotated in a direction perpendicular to the moving direction of the subject 10 and disposed to obtain more spatial information.

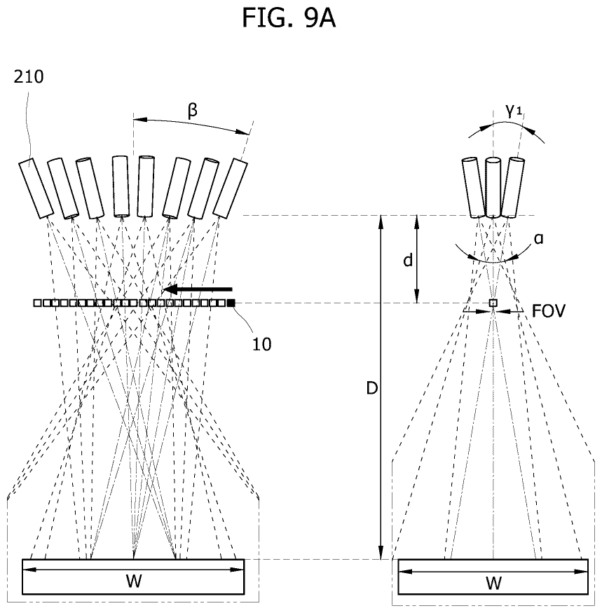

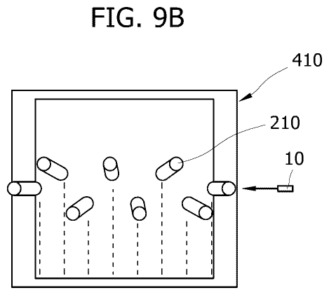

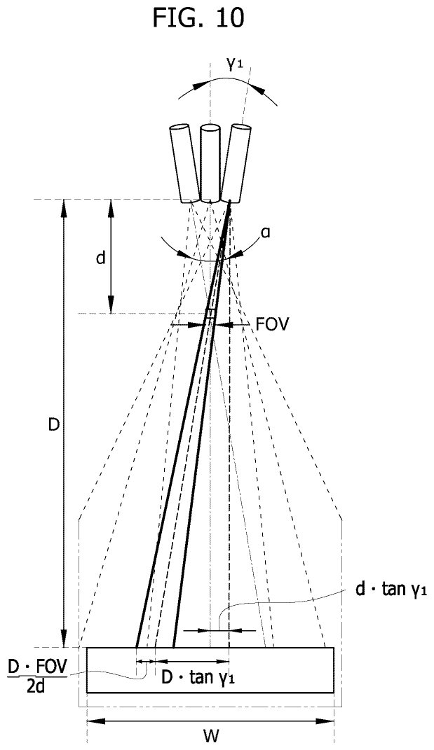

FIG. 9A is a side view for describing a spatial rotation arrangement of the X-ray sources according to one embodiment of the present invention. FIG. 9B is a plan view for describing the spatial rotation arrangement of the X-ray sources according to one embodiment of the present invention. FIG. 10 is an exemplary diagram for describing a second rotation angle according to one embodiment of the present invention.

Referring to FIGS. 9A and 9B, the spatial rotation arrangement may be applied to a structure in which the plurality of X-ray sources 210 disposed to be rotated by a first rotation angle β in a moving direction of the subject 10 and rotated by a second rotation angle γ in a direction perpendicular to the moving direction of the subject 10. In this case, focal points of the plurality of X-ray sources 210 may be disposed at equal intervals with respect to the moving direction of the subject 10, and a center of X-rays radiated by each X-ray source 210 may face the detection unit 400.

When the plurality of X-ray sources 210 are disposed in the spatial rotation arrangement, the X-ray sources 210 may be rotated in a direction perpendicular to the moving direction of the subject 10, thereby obtaining more spatial information. In this case, a radiation (emission) direction of each X-ray source 210 may face the detection unit 400, and a degree of rotation of the X-ray source in a direction perpendicular to the moving direction of the subject 10 may be determined within a range in which an X-ray transmission image of the subject 10 may be covered by the detection unit 400.

For example, the second rotation angle γ may be determined based on at least one of a magnification determined by an X-ray emission angle α, a distance d between the X-ray source and the subject, and a distance D-d between the subject and the detection unit, an FOV of the subject, and a width W of the detection unit. The X-ray emission angle α, the distance d between the X-ray source and the subject, the distance D-d between the subject and the detection unit, the FOV of the subject, and the width W of the detection unit may be as shown in FIG. 10. The larger the emission angle α and the width W (size) of the detection unit and the smaller the FOV of the subject and the magnification, the larger the second rotation angle γ may be. Therefore, the maximum value of the second rotation angle γ may be determined according to Expression 1 below.

$$tan γ \leq \frac{\frac{W}{2} - \frac{D \cdot FOV}{2 d}}{D - d} \qquad [ Expression 1 ]$$

In the case of the spatial rotation arrangement, as both the first rotation angle β and the second rotation angle γ are larger, an X-ray transmission image with a wider angle may be obtained, which is advantageous for 3D image synthesis. The first rotation angle β is a rotation angle with respect to a plane in the moving direction of the subject 10, and when an X-ray transmission image is obtained at a position far from a center of the detection unit, it is easy to obtain an X-ray transmission image in a relatively large angle range. However, in the case of the second rotation angle γ, since a position of the subject is fixed with respect to a rotation plane, when the area W of the detection unit is not wide, it is not easy to obtain an X-ray transmission image in a large angle range. Therefore, it is relatively difficult to obtain spatial information of an axis perpendicular to the moving direction of the subject.

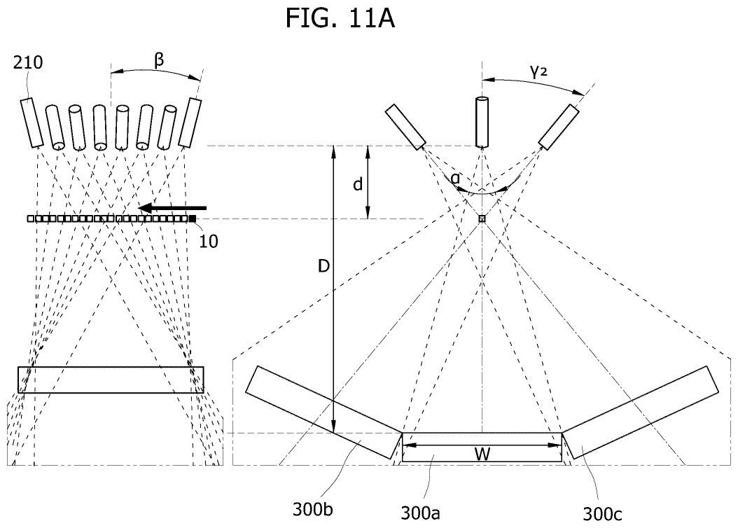

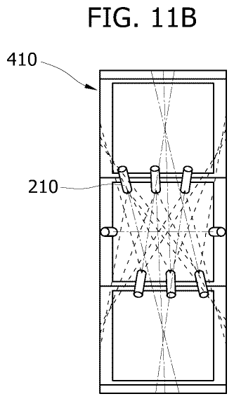

FIG. 11A is a side view for describing an enlarged spatial rotation arrangement of the X-ray sources according to one embodiment of the present invention. FIG. 11B is a plan view for describing the enlarged spatial rotation arrangement of the X-ray sources in one embodiment of the present invention.

Referring to FIGS. 11A and 11B, the enlarged space rotation arrangement may be applied to a structure in which the plurality of X-ray sources 210 are disposed to be rotated by a first rotation angle β in a moving direction of the subject 10 and rotated by a third rotation angle γ2 in a direction perpendicular to the moving direction of the subject 10, and a plurality of detection units 400a, 400b, and 400c are disposed in a direction perpendicular to the moving direction of the subject 10. In this case, the third rotation angle γ2 may have a larger value than the second rotation angle γ of the spatial rotation arrangement.

When the plurality of X-ray sources 210 and the plurality of detection units 400a, 400b, and 400c are disposed in the enlarged space rotation arrangement, a wide detection unit may be secured, thereby obtaining a wide rotation angle with respect to a plane perpendicular to the moving direction of the subject. In the enlarged space rotation arrangement, the number, arrangement position, and angle of the detection units may be changed according to the application purpose.

According to the present embodiment, a 2D or 3D image of a subject moving by a transfer unit 100 may be obtained in real time using a plurality of X-ray sources.

According to the present embodiment, by arranging a plurality of X-ray sources in at least one of a parallel arrangement, a planar rotation arrangement, a spatial rotation arrangement, and an enlarged spatial rotation arrangement, without rotation of a subject or a X-ray source-detection unit (sensor) combination, a 3D nondestructive inspection may be performed along a movement path of the subject during a production process.

As used herein, the term “unit” may include a unit implemented in hardware, software, or firmware, and may be used interchangeably with other terms, for example, logic, logic block, part, or circuitry. The unit may be a single integral component, or a minimum unit or part thereof, adapted to perform one or more functions. For example, according to one embodiment, the “unit” may be implemented in a form of an application-specific integrated circuit (ASIC).

Implementations described herein may be implemented in, for example, a method or process, an apparatus, a software program, a data stream, or a signal. Although discussed only in the context of a single form of implementation (e.g., discussed only as a method), implementations of the discussed features may also be implemented in other forms (for example, an apparatus or a program). The apparatus may be implemented in suitable hardware, software, firmware, and the like. A method may be implemented in an apparatus such as a processor, which is generally a computer, a microprocessor, an integrated circuit, a processing device including a programmable logic device, or the like. Processors also include communication devices such as a computer, a cell phone, a portable/personal digital assistant (“PDA”), and other devices that facilitate communication of information between end-users.

Although the present invention has been described with limited embodiments and drawings, the present invention is not limited to thereto, and instead, it would be appreciated by those skilled in the art that various modifications and changes may be made to these embodiments without departing from the principles and spirit of the present invention, the scope of which is defined by the claims and their equivalents.

What is claimed is:

1. An X-ray inspection apparatus comprising:
a transfer unit configured to transfer a subject in a first direction;
a plurality of X-ray sources arranged to be spaced apart from each other in a second direction intersecting the first direction and configured to radiate X-rays toward the subject;
an X-ray control unit configured to control an arrangement of the plurality of X-ray sources;
a detection unit positioned below the transfer unit and configured to detect an X-ray transmission image of the X-rays passing through the subject; and
an image processing unit configured to generate an X-ray image based on the X-ray transmission image detected by the detection unit.

2. The X-ray inspection apparatus of claim 1, wherein the plurality of X-ray sources are sequentially driven one by one under a control of the X-ray control unit to radiate the X-rays, and
the detection unit acquires the X-ray transmission image in synchronization with X-ray radiation of each X-ray source.

3. The X-ray inspection apparatus of claim 2, wherein the X-ray control unit controls an X-ray radiation time and an X-ray amount of each X-ray source.

4. The X-ray inspection apparatus of claim 1, wherein the X-ray control unit controls the plurality of X-ray sources to be disposed in at least one of a parallel arrangement, a plane rotation arrangement, a spatial rotation arrangement, and an enlarged spatial rotation arrangement.

5. The X-ray inspection apparatus of claim 4, wherein, in the parallel arrangement, the plurality of X-ray sources are disposed perpendicular to an X-ray detection surface of the detection unit and disposed parallel to each other at a set interval.

6. The X-ray inspection apparatus of claim 4, wherein, in the plane rotation arrangement, the plurality of X-ray sources are disposed perpendicular to an X-ray detection surface of the detection unit, and
each X-ray source is rotated to face a center of the X-ray detection surface.

7. The X-ray inspection apparatus of claim 4, wherein, in the spatial rotation arrangement, the plurality of X-ray sources are rotated by a first rotation angle in a moving direction of the subject and rotated by a second rotation angle in a direction perpendicular to the moving direction of the subject, focal points of the plurality of X-ray sources are disposed at equal intervals with respect to the moving direction of the subject, and a center of the X-rays radiated by each X-ray source is disposed to face an X-ray detection surface of the detection unit.

8. The X-ray inspection apparatus of claim 7, wherein the second rotation angle is determined by at least one of an X-ray emission angle, a distance between the X-ray source and the subject, a distance between the subject and the detection unit, and a field of view (FOV) of the subject, and an area of the detection unit.

9. The X-ray inspection apparatus of claim 7, wherein, in the enlarged space rotation arrangement, the plurality of X-ray sources are disposed to be rotated by the first rotation angle in the moving direction of the subject and rotated by a third rotation angle in the direction perpendicular to the moving direction of the subject, and a plurality of detection units are disposed in the direction perpendicular to the moving direction of the subject,
wherein the third rotation angle has a larger value than the second rotation angle.

10. The X-ray inspection apparatus of claim 1, wherein the plurality of X-ray sources operate in at least one of a stitching mode, a region of interest (ROI) mode, and a tomography mode.

11. The X-ray inspection apparatus of claim 10, wherein, when the plurality of X-ray sources operate in the stitching mode,
the plurality of X-ray sources are sequentially driven in the first direction as the subject moves in the first direction,
the detection unit detects a plurality of partial X-ray transmission images as the X-ray sources are sequentially driven, and
the image processing unit image-processes the plurality of partial X-ray transmission images detected by the detection unit to generate one or more high-resolution X-ray images.

12. The X-ray inspection apparatus of claim 10, wherein, when the plurality of X-ray sources operate in the ROI mode,
as the subject moves in the first direction, an X-ray source, to which the FOV of the subject reaches, among the plurality of X-ray sources, radiates the X-rays,
the detection unit detects an X-ray transmission image of the FOV, and
the image processing unit image-processes the X-ray transmission image of the FOV detected by the detection unit to generate one or more X-ray images of the FOV.

13. The X-ray inspection apparatus of claim 10, wherein, when the plurality of X-ray sources operate in the tomography mode,
the plurality of X-ray sources are sequentially driven in the first direction to radiate the X-rays at different angles a plurality of times as the subject moves in the first direction,
the detection unit detects X-ray transmission images at different angles, and
the image processing unit image-processes the X-ray transmission image detected through the detection unit to generate a tomogram.

14. An X-ray inspection method comprising:
arranging, by an X-ray control unit, a plurality of plurality of X-ray sources in an arrangement structure selected by a user;
sequentially driving the plurality of X-ray sources under a control of the X-ray control unit to radiate X-rays onto a subject moved by a transfer unit;
detecting, by a detection unit, an X-ray transmission image of the X-rays passing through the subject; and
generating, by an image processing unit, an X-ray image based on the X-ray transmission image detected by the detection unit.

15. The X-ray inspection method of claim 14, wherein, in the arranging of the plurality of X-ray sources, the X-ray control unit controls the plurality of X-ray sources to be disposed in at least one of a parallel arrangement, a plane rotation arrangement, a spatial rotation arrangement, and an enlarged spatial rotation arrangement.

16. The X-ray inspection method of claim 15, wherein, in the parallel arrangement, the plurality of X-ray sources are disposed perpendicular to an X-ray detection surface of the detection unit and disposed parallel to each other at a set interval.

17. The X-ray inspection method of claim 15, wherein, in the plane rotation arrangement, the plurality of X-ray sources are disposed perpendicular to an X-ray detection surface of the detection unit, and each X-ray source is rotated to face a center of the X-ray detection surface.

18. The X-ray inspection method of claim 15, wherein, in the spatial rotation arrangement, the plurality of X-ray sources are rotated by a first rotation angle in a moving direction of the subject and rotated by a second rotation angle in a direction perpendicular to the moving direction of the subject, focal points of the plurality of X-ray sources are disposed at equal intervals with respect to the moving direction of the subject, and a center of the X-rays radiated by each X-ray source is disposed to face an X-ray detection surface of the detection unit.

19. The X-ray inspection method of claim 18, wherein, in the enlarged space rotation arrangement, the plurality of X-ray sources are disposed to be rotated by the first rotation angle in the moving direction of the subject and rotated by a third rotation angle in the direction perpendicular to the moving direction of the subject, and a plurality of detection units are disposed in a direction perpendicular to the moving direction of the subject,
wherein the third rotation angle has a larger value than the second rotation angle.

20. The X-ray inspection method of claim 14, wherein, in the radiating of the X-rays, wherein the plurality of X-ray sources operate in at least one of a stitching mode, a region of interest (ROI) mode, and a tomography mode.
